(19) United States
(12) Patent Application Publication
(10) Pub. No.: US 2025/0020641 A1
(43) Pub. Date: Jan. 16, 2025

(54) LATERAL FLOW IMMUNOASSAY DEVICE FOR ALBUMIN DETECTION

(71) Applicants: PEKING UNIVERSITY, Beijing (CN); PEKING UNIVERSITY FIRST HOSPITAL, Beijing (CN)

(72) Inventors: Li YANG, Beijing (CN); Yue CUI, Beijing (CN)

(21) Appl. No.: 18/412,482

(22) Filed: Jan. 13, 2024

(30) Foreign Application Priority Data
Jul. 12, 2023 (CN) 2023108521970

(51) Int. Cl. G01N 33/543; G01N 33/68

(52) U.S. Cl. CPC G01N 33/54388; G01N 33/68; G01N 2333/765

(57) ABSTRACT

Disclosed is a lateral flow immunoassay device for albumin detection, including one or more strips that are formed by sequentially connecting the following components: a) a sample pad for receiving a sample to be tested; b) a conjugate pad for temporarily storing a gold nanoparticle labeled antibody; c) a test region for immobilizing an antigen, a nitrocellulose membrane being arranged above the test region; and d) an absorbent pad for increasing capillarity; two ends of the nitrocellulose membrane overlap with the conjugate pad and the absorbent pad at an overlapping length of 1 mm to 5 mm, respectively; and further including: e) a backing card for supporting the above components, the backing card being a transparent glass, an upper surface of the transparent glass being provided with the test region for immobilizing the antigen to capture the gold nanoparticle labeled antibody, and the transparent glass being pretreated by silanization.

### CROSS REFERENCE TO RELATED APPLICATION

This patent application claims the benefit and priority of Chinese Patent Application No. 2023108521970, filed with the China National Intellectual Property Administration on Jul. 12, 2023, the disclosure of which is incorporated by reference herein in its entirety as part of the present application.

### TECHNICAL FIELD

The present disclosure belongs to the technical field of albumin analysis, in particular relates to a lateral flow immunoassay (LFIA) device for albumin detection.

### BACKGROUND

Albumin analysis tools have been developed and used for decades. There are various methods for albumin analysis, such as enzyme-linked immunosorbent assay (ELISA), lateral flow immunoassay (LFIA), chemiluminescent enzyme immunoassay (CLEIA), surface plasmon resonance (SPR), time-resolved fluorescence (TRF), electrochemistry, capillary electrophoresis (CE), high-performance liquid chromatography (HPLC), and microfluidics. Among these detection methods, LFIA is considered to be one of the most promising tools for preclinical diagnosis due to its convenient operation process, low cost, and rapid response. The LFIA is not only one of the important research tools in the field of scientific research, but also generally used in people's daily life. For example, the most common early pregnancy test strips, blood glucose monitoring test strips, and rapid diagnostic kits for infectious diseases, as well as the rapid detection kits for novel coronavirus antigens and antibodies that emerged as a result of the novel coronavirus epidemic, are all based on a principle of the LFIA.

LFIA is a simple and rapid immunological detection technology based on the combination of immunological technology and chromatographic technology, and appeared in the early 1980s.

In the LFIA, a specific antigen is immobilized on a nitrocellulose membrane (NC membrane) in the form of a strip, and another labeled antibody is adsorbed on a conjugate pad. When an analyte is added to a sample pad at one end of a test strip, the analyte moves forward through capillary action, and dissolves and reacts with a labeled antibody on the conjugate pad; a resulting antigen bounded gold nanoparticle labeled antibody conjugate moves to a test region where an antigen is immobilized, specifically binds to the antigen in the test region, and then is retained and concentrated on the test region. Thus, intuitive results can be obtained by the labeled antibody which is visually observable. The captured antigen protein immobilization on a detection device is a key step in the albumin analyte detection, and can determine the detection concentration range, limit of detection (LOD), detection rate, and detection sensitivity of a sample.

However, traditional paper-based LFIA generally shows the following defects:
1) A captured antigen is immobilized on a nitrocellulose membrane that is immobilized on a white substrate. However, both the nitrocellulose membrane and the substrate have opacity, which is not conducive to the detection of optical signals.
2) Colored lateral flow markers are represented by colloidal gold nanoparticles. The colloidal gold nanoparticles is a kind of nanoparticle with a surface electric double layer structure formed by chloroauric acid under the action of a reducing agent, and has simple preparation process, stable performance, and desirable biocompatibility. The colloidal gold nanoparticles can be combined with proteins and nucleic acids through non-covalent bonds. Moreover, the colloidal gold nanoparticles have a simple and rapid labeling process, thereby receiving extensive attention and application in the field of rapid detection. However, the colloidal gold nanoparticles still result in a poor sensitivity for the detection of low-concentration samples, and requires a relatively high raw material costs in the preparation process.
3) In the LFIA, the sample flows and passes through a membrane driven by capillary force. A pore size of the absorbent material/membrane and a viscosity of the sample are two parameters that have a direct influence on the flow rate of the sample when passing through a system; flow rate/velocity is a key factor affecting the detection efficiency. It is of great significance to improve the detection efficiency in the case of a large-scale influenza epidemic.

Currently, due to the above-mentioned technical problems, most LFIA techniques can only be used for qualitative diagnosis. In view of this, it is a future development trend to improve the sensitivity of the LFIA and to conduct quantitative analysis on detection substances.

### SUMMARY

In view of the technical problems of 1) to 3) described above, the present disclosure provides a LFIA device for albumin detection.

To solve the above technical problems, the present disclosure provides the following technical solutions.

The present disclosure provides a LFIA device for albumin detection, including one or more strips that are formed by sequentially connecting the following components:
a) a sample pad for receiving a sample to be tested;
b) a conjugate pad for temporarily storing a gold nanoparticle labeled antibody;
c) a test region for immobilizing an antigen, where a nitrocellulose membrane is arranged above the test region, and
d) an absorbent pad for increasing capillarity;
where two ends of the nitrocellulose membrane overlap with the conjugate pad and the absorbent pad at an overlapping length of 1 mm to 5 mm, respectively; and
further includes e) a backing card for supporting the above components, where the backing card is a transparent glass, an upper surface of the transparent glass is provided with the test region for immobilizing the antigen to capture the gold nanoparticle labeled antibody, and the transparent glass is pretreated by silanization.

In some embodiments, the sample pad is mainly used for loading a sample, and can be prepared from a polyester fiber membrane or a glass fiber membrane.

In some embodiments, the conjugate pad for temporarily storing the gold nanoparticle labeled antibody is generally prepared from a glass fiber, a non-woven fabric, or a polyester fiber with desirable water absorption and uniformity. According to different usage requirements, those skilled in the art could adjust the material, thickness, release speed, and pre-immersion treatment of the conjugate pad to ensure a binding stability of a conjugate and continuous and uniform migration to an area used to immobilize the antibody.

In some embodiments, the test region has a width of 1 mm to 5 mm and a length of 2 mm to 30 mm. In some embodiments, the test region has a size of 1 mm×30 mm, namely a width of 1 mm and a length of 30 mm.

In some embodiments, the absorbent pad for increasing capillary is mainly used for absorbing a waste liquid, and is generally prepared from an absorbent paper with a stable property, a large water absorption capacity, and a desirable water absorption performance.

In some embodiments, a reagent used for the silanization is 3-aminopropyltriethoxysilane.

In some embodiments, the silanization of the transparent glass includes: ultrasonically treating a glass substrate in water at 100 W to 150 W and 40 KHz for 10 min to 30 min to obtain an ultrasonically treated glass substrate; rinsing the ultrasonically treated glass substrate with ultrapure water and drying in nitrogen sequentially to obtain a dried glass substrate, and treating the dried glass substrate in an ultraviolet ozone cleaner or an oxygen plasma for 10 min to 30 min to remove organic pollutants to obtain a clean glass substrate; immersing the clean glass substrate in a freshly-prepared silane coupling agent solution at a temperature of 90° C. to 120° C. for 1 h to 2 h to obtain an immersed glass substrate, where the silane coupling agent solution is prepared by dissolving a silane coupling agent in anhydrous toluene at a concentration of 1% to 10% by volume; and washing the immersed glass substrate thoroughly with ethanol and drying at ambient temperature before use.

In some embodiments, the silanization of the transparent glass specifically is performed by: ultrasonically treating the glass substrate in water at 120 W and 40 KHz for 10 min to obtain the ultrasonically treated glass substrate; rinsing the ultrasonically treated glass substrate with ultrapure water and drying in nitrogen sequentially to obtain the dried glass substrate, and treating the dried glass substrate in the ultraviolet ozone cleaner for 20 min to remove the organic pollutants to obtain the clean glass substrate; immersing the clean glass substrate in the freshly-prepared silane coupling agent solution at 110° C. for 2 h to obtain the immersed glass substrate, where the silane coupling agent solution is prepared by dissolving the silane coupling agent in anhydrous toluene at 5% by volume; and washing the immersed glass substrate thoroughly with ethanol and drying at ambient temperature before use.

In some embodiments, the LFIA device for albumin detection is also used in detection fields of clinical medical disease detection, biomedicine, environment detection, and food sanitation.

The present disclosure has the following technical effects:
1) The present disclosure provides a LFIA device for albumin detection and use thereof. The inventors have surprisingly found that in the traditional paper-based LFIA, a antigen layer is sprayed on a nitrocellulose membrane, and a three-dimensional structure of the membrane causes uneven distribution of colloidal gold nanoparticles. This defect can be solved in glass-based LFIA. This is because the immobilization occurs on a two-dimensional plane, and an antigen-bound liquid is evenly distributed on a glass surface, thereby reducing the distribution of the bounded colloidal gold nanoparticles per unit area, and reducing a detection concentration and a LOD, such that sample detection can be completed at a LOD of 10 ng/mL. Meanwhile, a cost of the LFIA device for albumin detection can also be reduced.
2) In the traditional paper-based LFIA, an antigen bounded colloidal gold nanoparticle antibody conjugate moves within the nitrocellulose membrane to reach the test line, which limits a moving speed of the conjugate and prolongs a time of the entire detection. However, based on the improvement by the technical solutions of the present disclosure, in the glass-based LFIA, the conjugate can pass through the nitrocellulose membrane, and also can pass through an overlapping space between the glass and the nitrocellulose membrane, such as the gap shown in FIG. 1. Here, the limitation of movement is greatly reduced, such that a detection efficiency can be improved. An enormous improvement in the detection efficiency brings great advantages for future commercial applications, and is advantageous in pandemic situations where highly efficient detection is required for initial screening of diseases. For example, shortening a nucleic acid detection time for the novel coronavirus epidemic is beneficial to urban management.

In the present disclosure, an existing polyvinyl chloride (PVC) plastic backing card is replaced with a transparent glass substrate, and silanization modification is conducted on a part of the transparent glass substrate for immobilizing an antigen. The LFIA device for albumin detection, which replaces preparation of an immobilized antigen on a nitrocellulose membrane, can solve technical problems recorded in the prior art during LFIA. The LFIA device shows technical effects, such as improving a detection concentration range and a LOD of a low-concentration albumin sample, improving a detection sensitivity and a detection efficiency, reducing a dosage of colloidal gold nanoparticles, and enhancing the albumin biological detection. Compared with the prior art, the LFIA device of the present disclosure has achieved corresponding unexpected technical effects.

### BRIEF DESCRIPTION OF THE DRAWINGS

To describe the technical solutions in the embodiments of the present disclosure or in the prior art more clearly, the following briefly describes the accompanying drawings required for describing the embodiments or the prior art. Apparently, the accompanying drawings in the following description only show some embodiments of the present disclosure, and a person of ordinary skill in the art may still derive other drawings from these accompanying drawings without creative efforts.

FIG. 1 shows a structural schematic diagram of the LFIA device according to an embodiment of the present disclosure.

FIG. 2 shows a structural schematic diagram of a LFIA device in the prior art.

FIG. 3 shows a schematic diagram for signal display of the LFIA device according to an embodiment of the present disclosure.

FIGS. 4A-4B show detection results of a glass-based LFIA; where FIG. 4A is a photographic picture, and FIG. 4B is a picture under an optical microscope (an upper line represents a test line, a lower line represents a control line, and albumin sample solutions have concentrations from left to right as follows: 0 ng/ml, 1 ng/mL, 2 ng/mL, 4 ng/mL, 6 ng/mL, 8 ng/mL, 10 ng/ml, 100 ng/mL, 200 ng/ml, 800 ng/mL, 10 μg/mL, 40 μg/mL, 80 μg/mL, and 100 μg/mL).

FIG. 5 shows a calibration curve of albumin detected with the LFIA according to an embodiment of the present disclosure.

FIG. 6 shows a detection time in detecting albumin of different concentrations with the LFIA according to an embodiment of the present disclosure.

FIG. 7 shows a comparison result of a specificity in detecting albumin with the LFIA according to an embodiment of the present disclosure (comparison with different proteins including neutrophil gelatinase associated lipocalin (NGAL), human chorionic gonadotropin (HCG), cardiac troponin I (CTNI), and kidney injury molecule 1 (KIM-1)).

FIG. 8 shows a time stability of albumin detected with the LFIA according to an embodiment of the present disclosure.

FIG. 9 shows a comparison curve between detection results of albumin in clinical patient samples by the LFIA according to an embodiment of the present disclosure and clinical detection results of hospitals.

FIG. 10 shows a test paper photo for detecting albumin in clinical patient samples with the LFIA according to an embodiment of the present disclosure.

FIG. 11 shows a comparison data between detection results of albumin in clinical patient samples by the LFIA according to an embodiment of the present disclosure and clinical detection results of hospitals.

FIG. 12 shows a detection picture of traditional paper-based LFIA (where albumin sample solutions have concentrations from left to right as follows: 10 ng/mL, 100 ng/mL, 200 ng/mL, 600 ng/ml, 1 μg/mL, 10 μg/mL, 20 μg/mL, 40 μg/mL, 60 μg/mL, 80 μg/mL, 100 μg/mL, 200 μg/mL, 400 μg/mL, 600 μg/mL, 800 μg/mL, and 1 mg/mL).

FIG. 13 shows a detection curve of traditional paper-based LFIA.

FIG. 14 shows a detection time of a traditional detection test paper for albumin at different concentrations.

FIGS. 15A and 15B show the distribution of gold nanoparticles, where FIG. 15A shows the distribution of gold nanoparticles on a paper substrate, and FIG. 15B shows the distribution of gold nanoparticles on a glass substrate.

Reference numerals: 1-sample pad, 2-conjugate pad, 3-nitrocellulose membrane, 4-absorbent pad, 5-test line, 6-control line, 7-transparent glass, 8-sample pad, 9-conjugate pad, 10-absorbent pad, 11-PVC substrate, 12-test line, 13-control line, and 14-nitrocellulose membrane.

### DETAILED DESCRIPTION

To enable those skilled in the art to better understand the technical solutions of the present disclosure, the present disclosure will be further described in detail below with reference to the accompanying drawings and specific examples.

### Example 1

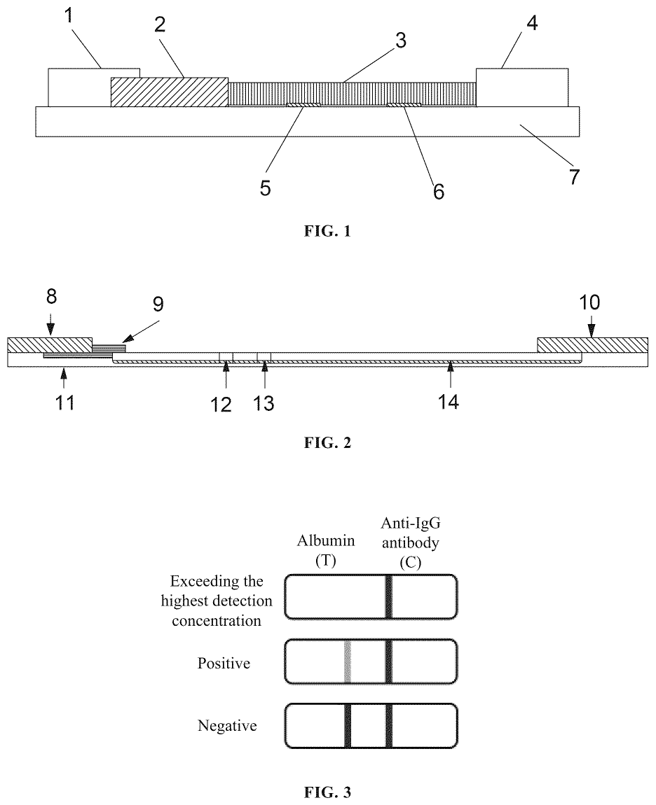

This example provided a LFIA device for albumin detection. As shown in FIG. 1, a sample pad, a conjugate pad, a nitrocellulose membrane, and an absorbent pad were mounted on a backing card (1 mm×60 mm) for supporting the above components. The backing card had a test region for immobilizing an antigen, and the backing card was a transparent glass treated by silanization. The test region was set at a center of the backing card, the nitrocellulose membrane was set above the test region, and two ends of the nitrocellulose membrane overlapped with the conjugate pad and the absorbent pad at an overlapping length of 2 mm, respectively. There was a gap between the nitrocellulose membrane and the transparent glass backing card, and the transparent glass had a test line. Albumin (0.8 mg/mL) was dispensed onto the test line on the glass surface at a volume ratio of 2 μL/cm; goat anti-mouse IgG (1 mg/mL) was chosen as a control line to capture a gold nanoparticle labeled antibody and dispensed onto the glass surface at a volume of 2 μL/cm and at a distance of 4 mm from the test line. When an obtained test strip was attached to albumin with a self-designed 3D printed clip for testing, an albumin solution was gently dropped on the sample pad, and the test strip was dried at ambient temperature for 30 min. As shown in FIG. 3, according to the results seen from the test line and the control line based on a competition reaction: a dark color on the T line (test line) represents a negative result, indicating that there is no albumin analyte in the test liquid. A light color on the T line (test line) represents a positive result, indicating that there is albumin analyte in the test liquid.

A specific modification method of the test region was performed as follows: a glass substrate was ultrasonically treated in water at 120 W and 40 KHz for 10 min. The ultrasonically treated glass substrate was rinsed with ultrapure water and dried in nitrogen sequentially, and then treated in an ultraviolet ozone cleaner for 20 min to remove organic pollutants to obtain a clean glass substrate. The clean glass substrate was immersed in a freshly-prepared 3-aminopropyltriethoxysilane solution (dissolved in anhydrous toluene at 5% by volume) at 110° C. for 2 h. The immersed glass substrate was washed thoroughly with ethanol and dried at ambient temperature before use.

### Comparative Example 1

This example provided a LFIA device for albumin detection. As shown in FIG. 2, a sample pad, a conjugate pad, a nitrocellulose membrane, and an absorbent pad were mounted on a PVC backing card (1 mm×60 mm) for assembling a LFIA test strip. The nitrocellulose membrane was placed at a center of the PVC backing card. The conjugate pad containing colloidal gold nanoparticle labeled anti-albumin antibody was attached to the nitrocellulose membrane with an overlap of 2 mm. The absorbent pad was attached to the other side of the nitrocellulose membrane with an overlap of 2 mm, and the sample pad was placed on the conjugate pad. Albumin (0.8 mg/mL) was immobilized onto the nitrocellulose membrane at a volume ratio of 2 μL/cm as the test line. Goat anti-mouse IgG (1 mg/mL) was chosen as a control line capture antibody and dispensed onto the nitrocellulose membrane at a volume of 2 μL/cm and at a distance of 4 mm from the test line. An obtained test strip was attached to the albumin chip with a self-designed 3D printed clip for testing. An albumin solution was gently dropped on the sample pad, and a test stroke was dried at ambient temperature for 30 min. The results seen from the test line and control line were observed and analyzed through the nitrocellulose membrane afterwards.

In the present disclosure, the test devices according to Example 1 and Comparative Example 1 were separately tested in aspects of LOD, detection concentration, response time, and colloidal gold nanoparticles distribution. The specific effects and comparisons were as follows:

### 1) Comparison of LOD for Samples

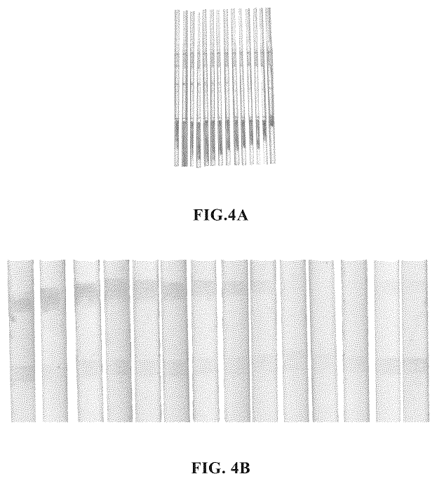

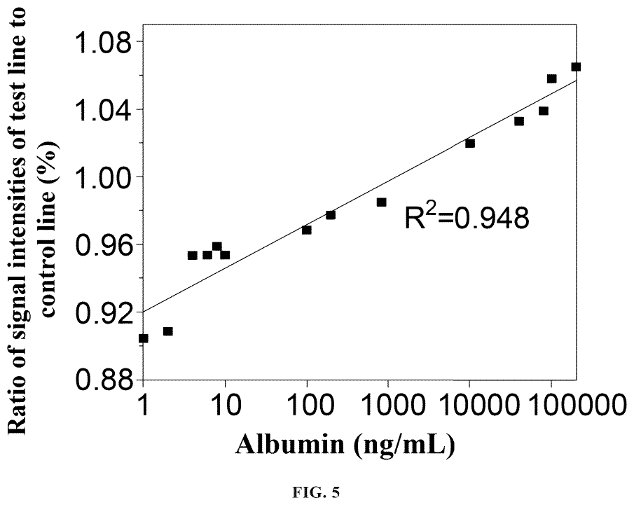

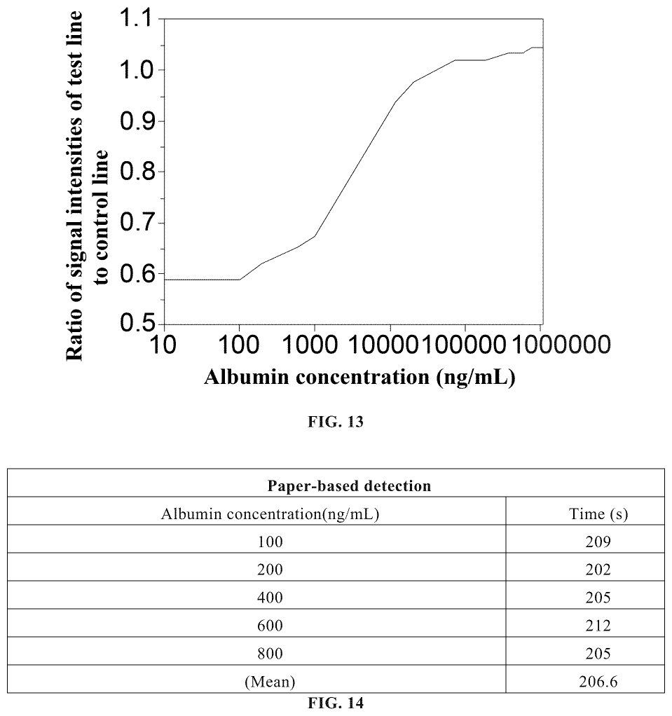

As shown in FIGS. 4A-4B, the detection performances of glass-based LFIA and paper-based LFIA were compared by analyzing the results of a series of albumin solutions with different concentrations. A competitive assay was used in this process and the results are interpreted in FIGS. 4A-4B. The color of the test line in the figure decreases as the concentration of albumin in the sample solution increases, and a root cause of this phenomenon is the principle of competitive detection. When fewer sites are available on the colloidal gold nanoparticles anti-albumin antibody conjugates, less colloidal gold nanoparticles are captured by albumin on the test line. This occurs when a large amount of albumin molecules in the sample occupy the vacancies on the conjugates. As shown in panel (a) and panel (b) of FIG. 4, when testing with the glass-based LFIA, a color of the test line becomes lighter when the albumin concentration increases, and disappears in the 100 μg/mL albumin solution. FIG. 5 shows a calibration curve of albumin detected with the LFIA, with a LOD of 10 ng/mL. A signal of the calibration curve comes from ImageJ, and the darker the color is, the smaller the ImageJ signal is (where a black signal is set to 0, and a white signal is set to 255). As shown in FIG. 13, the LOD of the paper-based LFIA is found to be 100 ng/ml, which is 1000% higher than that of the glass-based LFIA. Therefore, the glass-based LFIA exhibits a lower value of LOD in experiments, and is advantageous in real-time detection.

### 2) Comparison of Detection Concentrations for Samples

The data for the paper-based LFIA is shown in FIG. 13, where the detection signals increase dramatically in the concentration range from 100 ng/ml to 100 μg/mL. This large jump in concentration levels from 1 μg/mL to 10 μg/mL reduces a detection range, making it impossible to establish a regression equation over the entire analytical range. Furthermore, the paper-based LFIA shows convincing detection results at albumin concentrations ranging from 100 ng/mL to 100 μg/mL. However, the paper-based LFIA is unable to distinguish concentration changes of albumin solutions when the albumin concentration is below its LOD of 100 ng/mL. The glass-based LFIA solves the above-mentioned problems, increases the detection sensitivity to a limit of 10 ng/mL, and extends the detection range to 10 ng/mL-100 μg/mL, thereby reducing the LOD by 10 times. In conclusion, the glass-based LFIA has advantages in detection range and LOD value, and is extremely promising in practical product application.

### 3) Comparison of Response Time for Samples

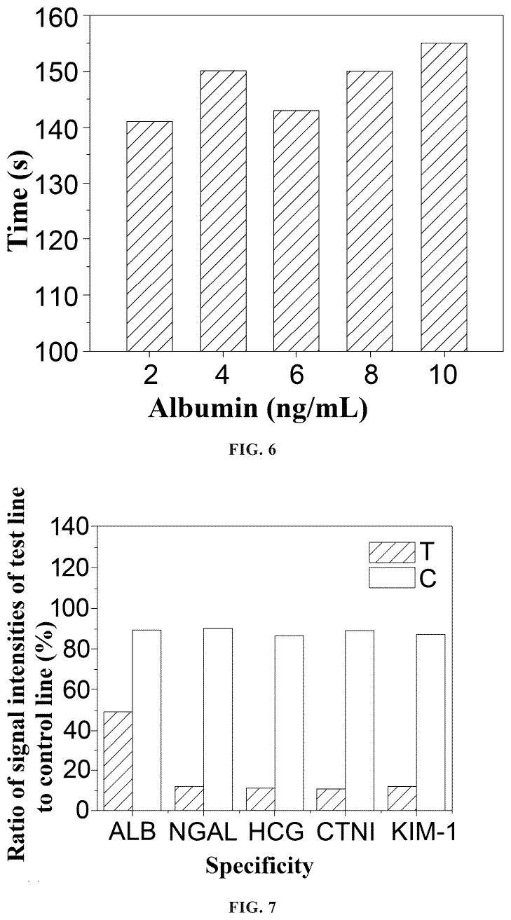

Another advantage of detection based on the glass-based LFIA testing is its high detection efficiency. In the traditional paper-based LFIA, an analyte moves within the nitrocellulose membrane to reach a test line, thereby limiting a moving speed of the analyte and prolonging a time of the entire detection. However, in the glass-based LFIA, an analyte could pass through both the nitrocellulose membrane and an intersecting space between the glass and the nitrocellulose membrane, where the limit of movement was greatly reduced. FIG. 6 and FIG. 14 show the detailed flow time for the two methods at different albumin concentrations. The glass-based method has an average detection time of 147.8 s, while the paper-based method has an average detection time of 206.6 s, indicating an increase in detection efficiency of about 28.5%. The huge improvement in detection efficiency brings great potential for future commercial applications, and is advantageous in pandemic situations where highly efficient detection is required for initial screening of diseases.

### 4) Selectivity and Stability of LFIA

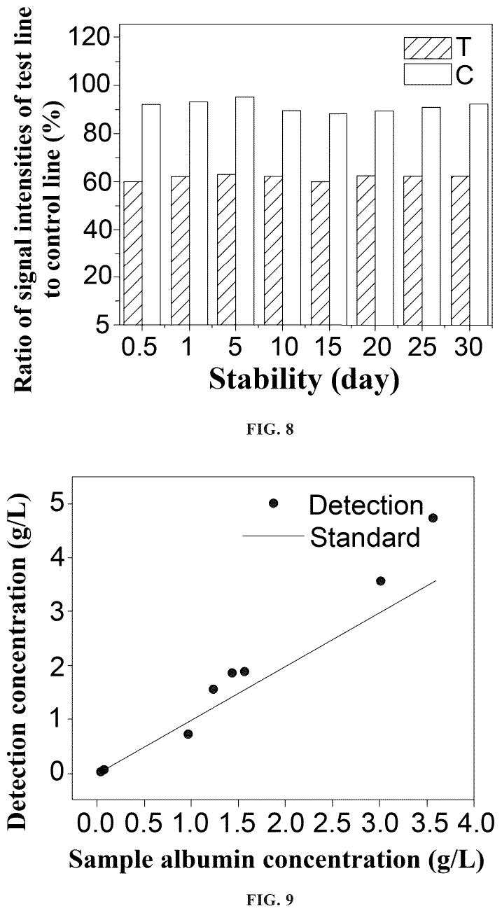

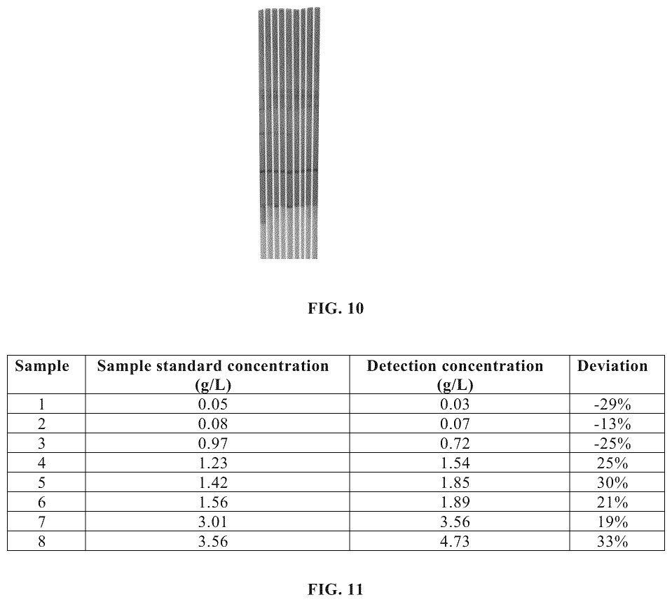

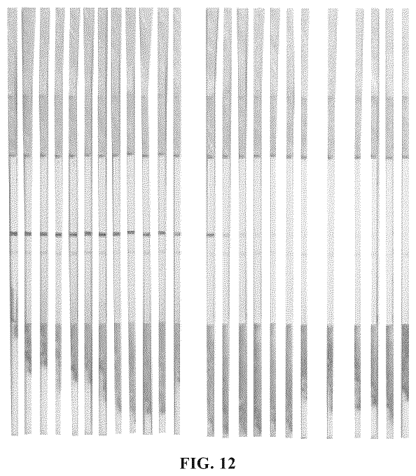

A cross-reactivity of the LFIA was tested by dropping different analytes NGAL, HCG, CTNI, and KIM-1 on a test strip. The presence or absence of signals on the T line and C line could be observed to determine whether there is a non-specific reaction. When NGAL, HCG, CTNI, and KIM-1 were added to the LFIA, there was no test signal, indicating that LFIA has a desirable specificity, as shown in FIG. 8.

In terms of stability, the glass-LFIA test strip was stored in a dark and dry environment at ambient temperature, and it was observed whether the signal intensity changed after 30 days. As shown in FIG. 9, the signal remains unchanged within 30 days, indicating that the glass-LFIA test strip has desirable selectivity and stability.

### 5) Distribution of Gold Nanoparticles

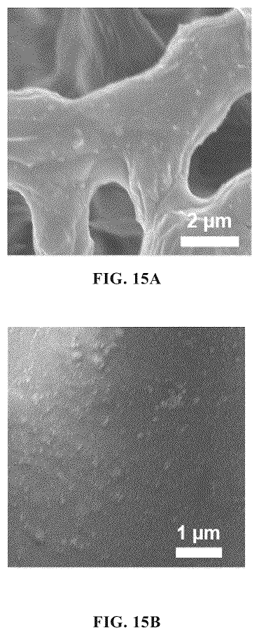

As shown in the left side of FIG. 15A, it can be seen in the results of the electron microscope image that: the fiber has an inhomogeneous shape, with depressed and raised areas in its cross-section, thus having different potentials to attach gold nanoparticles. The right side of FIG. 15A shows a distribution pattern of gold nanoparticles in the paper-based LFIA, where the colloidal gold nanoparticles are mainly located in a convex part of the cross-section. However, as shown on the right side of FIG. 15B, the gold nanoparticles located in the glass flake generate a uniform distribution pattern. The plate-like distribution of an antibody-gold conjugates is also advantageous for the antigen detection, since each antibody has an approximate probability of binding to a target protein. Therefore, immobilizing the antigen from the nitrocellulose membrane to the glass surface improves the distribution of nanoparticles and reduces the dosage of gold nanoparticles, thereby reducing the cost.

It can be understood that the above embodiments are merely exemplary embodiments used to illustrate the principle of the present disclosure, and the present disclosure is not limited thereto. Various modifications and improvements can be made by those of ordinary skill in the art without departing from the spirit and essence of the present disclosure, and these modifications and improvements shall also be considered as falling within the scope of the present disclosure.

What is claimed is:

1. A lateral flow immunoassay (LFIA) device for albumin detection, comprising one or more strips that are formed by sequentially connecting the following components:
a) a sample pad for receiving a sample to be tested;
b) a conjugate pad for temporarily storing a gold nanoparticle labeled antibody;
c) a test region for immobilizing an antigen, wherein a nitrocellulose membrane is arranged above the test region; and
d) an absorbent pad for increasing capillarity;
wherein two ends of the nitrocellulose membrane overlap with the conjugate pad and the absorbent pad at an overlapping length of 1 mm to 5 mm, respectively; and
further comprising: e) a backing card for supporting the above components, wherein the backing card is a transparent glass, an upper surface of the transparent glass is provided with the test region for immobilizing the antigen to capture the gold nanoparticle labeled antibody, and the transparent glass is pretreated by silanization.

2. The LFIA device for albumin detection of claim 1, wherein the sample pad is selected from the group consisting of a polyester fiber membrane and a glass fiber membrane.

3. The LFIA device for albumin detection of claim 1, wherein the conjugate pad for temporarily storing the gold nanoparticle labeled antibody is prepared from one or more selected from the group consisting of a glass fiber, a non-woven fabric, and a polyester fiber.

4. The LFIA device for albumin detection of claim 1, wherein the test region has a width of 1 mm to 5 mm and a length of 2 mm to 30 mm.

5. The LFIA device for albumin detection of claim 1, wherein the absorbent pad for increasing capillary is mainly used for absorbing a waste liquid, and is prepared from an absorbent paper with a stable property, a large water absorption capacity, and a desirable water absorption performance.

6. The LFIA device for albumin detection of claim 1, wherein a reagent used for the silanization is 3-aminopropyltriethoxysilane.

7. The LFIA device for albumin detection of claim 1, wherein the silanization of the transparent glass comprises: ultrasonically treating a glass substrate in water at 100 W to 150 W and 40 KHz for 10 min to 30 min to obtain an ultrasonically treated glass substrate; rinsing the ultrasonically treated glass substrate with ultrapure water and drying in nitrogen sequentially to obtain a dried glass substrate, and treating the dried glass substrate in an ultraviolet ozone cleaner or an oxygen plasma for 10 min to 30 min to remove organic pollutants to obtain a clean glass substrate; immersing the clean glass substrate in a freshly-prepared silane coupling agent solution at 90° C. to 120° C. for 1 h to 2 h to obtain an immersed glass substrate, wherein the silane coupling agent solution is prepared by dissolving a silane coupling agent in anhydrous toluene at a concentration of 1% to 10% by volume; and washing the immersed glass substrate thoroughly with ethanol and drying at ambient temperature before use.

8. The LFIA device for albumin detection of claim 7, wherein the silanization of the transparent glass is performed by: ultrasonically treating the glass substrate in water at 120 W and 40 KHz for 10 min to obtain the ultrasonically treated glass substrate; rinsing the ultrasonically treated glass substrate with ultrapure water and drying in nitrogen sequentially to obtain the dried glass substrate, and treating the dried glass substrate in the ultraviolet ozone cleaner for 20 min to remove the organic pollutants to obtain the clean glass substrate; immersing the clean glass substrate in the freshly-prepared silane coupling agent solution at 110° C. for 2 h to obtain the immersed glass substrate, wherein the silane coupling agent solution is prepared by dissolving the silane coupling agent in anhydrous toluene at 5% by volume; and washing the immersed glass substrate thoroughly with ethanol and drying at ambient temperature before use.

9. The LFIA device for albumin detection of claim 1, wherein compared with a paper-based LFIA device having a limit of detection (LOD) of 100 ng/ml, the LFIA device for albumin detection has a LOD reduced to 10 ng/mL and a detection time shortened by 28.5%.

10. The LFIA device for albumin detection of claim 1, wherein the LFIA device for albumin detection is also used in detection fields of clinical medical disease detection, biomedicine, environment detection, and food sanitation.

11. The LFIA device for albumin detection of claim 9, wherein the sample pad is selected from the group consisting of a polyester fiber membrane and a glass fiber membrane.

12. The LFIA device for albumin detection of claim 9, wherein the conjugate pad for temporarily storing the gold nanoparticle labeled antibody is prepared from one or more selected from the group consisting of a glass fiber, a non-woven fabric, and a polyester fiber.

13. The LFIA device for albumin detection of claim 9, wherein the test region has a width of 1 mm to 5 mm and a length of 2 mm to 30 mm.

14. The LFIA device for albumin detection of claim 9, wherein the absorbent pad for increasing capillary is mainly used for absorbing a waste liquid, and is prepared from an absorbent paper with a stable property, a large water absorption capacity, and a desirable water absorption performance.

15. The LFIA device for albumin detection of claim 9, wherein a reagent used for the silanization is 3-aminopropyltriethoxysilane.

16. The LFIA device for albumin detection of claim 9, wherein the silanization of the transparent glass comprises: ultrasonically treating a glass substrate in water at 100 W to 150 W and 40 KHz for 10 min to 30 min to obtain an ultrasonically treated glass substrate; rinsing the ultrasonically treated glass substrate with ultrapure water and drying in nitrogen sequentially to obtain a dried glass substrate, and treating the dried glass substrate in an ultraviolet ozone cleaner or an oxygen plasma for 10 min to 30 min to remove organic pollutants to obtain a clean glass substrate; immersing the clean glass substrate in a freshly-prepared silane coupling agent solution at 90° C. to 120° C. for 1 h to 2 h to obtain an immersed glass substrate, wherein the silane coupling agent solution is prepared by dissolving a silane coupling agent in anhydrous toluene at a concentration of 1% to 10% by volume; and washing the immersed glass substrate thoroughly with ethanol and drying at ambient temperature before use.

17. The LFIA device for albumin detection of claim 16, wherein the silanization of the transparent glass is performed by: ultrasonically treating the glass substrate in water at 120 W and 40 KHz for 10 min to obtain the ultrasonically treated glass substrate; rinsing the ultrasonically treated glass substrate with ultrapure water and drying in nitrogen sequentially to obtain the dried glass substrate, and treating the dried glass substrate in the ultraviolet ozone cleaner for 20 min to remove the organic pollutants to obtain the clean glass substrate; immersing the clean glass substrate in the freshly-prepared silane coupling agent solution at 110° C. for 2 h to obtain the immersed glass substrate, wherein the silane coupling agent solution is prepared by dissolving the silane coupling agent in anhydrous toluene at 5% by volume; and washing the immersed glass substrate thoroughly with ethanol and drying at ambient temperature before use.

18. The LFIA device for albumin detection of claim 10, wherein the sample pad is selected from the group consisting of a polyester fiber membrane and a glass fiber membrane.

19. The LFIA device for albumin detection of claim 10, wherein the conjugate pad for temporarily storing the gold nanoparticle labeled antibody is prepared from one or more selected from the group consisting of a glass fiber, a non-woven fabric, and a polyester fiber.

20. The LFIA device for albumin detection of claim 10, wherein the test region has a width of 1 mm to 5 mm and a length of 2 mm to 30 mm.
